(19) United States
(12) Patent Application Publication
(10) Pub. No.: US 2025/0332068 A1
(43) Pub. Date: Oct. 30, 2025

(54) FILLING AID ASSEMBLY

(71) Applicant: PharmaSens AG, Biel-Benken (CH)

(72) Inventors: Scott MARTIN, Dunbar (GB); James MCLUSKY, Edinburgh (GB); Peter FISCHBACHER, Edinburgh (GB); Caroline MACKIE, Edinburgh (GB); Nicole BERNINI, 3550 (CH); Marcel BOTH, Kirchlindach (CH); Serge HÜGLI, Kirchberg (CH); Gilbert SCHILTGES, Bellach (CH); David TEUTSCH, Detligen (CH); Andrea SCHÜTZ FRIKART, Gümligen (CH)

(73) Assignee: PharmaSens AG, Biel-Benken (CH)

(21) Appl. No.: 18/871,541

(22) PCT Filed: Jun. 17, 2022
(86) PCT No.: PCT/EP2022/066589

§ 371 Date: Dec. 4, 2024

(51) Int. Cl. A61J 1/20; A61J 1/14; A61M 5/142

(52) U.S. Cl. CPC A61J 1/2089; A61J 1/1412; A61J 1/2065; A61M 5/14248

(57) ABSTRACT

A filling aid assembly (1) for filling a liquid into a reservoir (101) of a patch pump (100) comprising a piston (102) arranged within the reservoir (101) comprises a lid part (2) configured to be arranged on top of the patch pump (100), the lid part having an opening (21) for accessing the reservoir when the lid part (2) is arranged on top of the patch pump (100) to enable fluidic exchange between the reservoir (101) and a pre-filled container (10) containing the liquid. The filling aid assembly further comprises a dial ring (4) and a transmission gear assembly (5) that is in engagement with an internal gearing (41) of the dial ring (4) to translate a rotational movement of the dial ring (4) into a displacement of the piston (102) within the reservoir (101).

### TECHNICAL FIELD

The present invention relates to a filling aid assembly for filling a liquid into a reservoir of a patch pump and to a medical assembly comprising said filling aid assembly.

### PRIOR ART

Certain medical conditions, such as for instance diabetes, require regular administration of a liquid, such as a solution comprising insulin, into a patient's body through the patient's skin. To facilitate administration, patch-like medical devices are known, which are configured to be applied to the patient's skin and preferably to be worn by the patient over an extended period of time. Such patch-like devices generally comprise a hollow needle (cannula) configured to pierce the patient's skin and through which needle the liquid substance is being delivered into the patient's body. In particular if the liquid substance is to be administered slowly and/or regularly over an extended period of time, the needle is advantageously connected to a delivery system comprising an automatic pump mechanism, wherein the pump mechanism is housed within the patch-like medical device. In particular, the pump mechanism usually comprises a reservoir containing the liquid, the needle being ideally directly connected to said reservoir.

Different approaches are known in the art to initially fill the reservoir and/or to re-fill the reservoir with the liquid to be administered. A first approach consists of using pre-filled cartridges that are directly inserted into the patch-like medical device.

Another approach consists of having a syringe-like reservoir that is removable from the patch-like medical device and that comprises an input adapter for a vial, so that a vial may be attached to the reservoir once it has been taken out of the patch. The reservoir in such a case contains a piston that may be manually actuated to suck the liquid out of the vial once the vial is placed in the adapter. This approach has the disadvantage that the reservoir needs to be removed from the patch-like medical device for re-filling, which may increase the risk of contamination. In addition, the reservoir and the mechanism for actuating the piston need to be built in a rugged manner to avoid damage or wear-out when taking the reservoir out, manually actuating the piston and plugging the reservoir back into the patch-like medical device many times.

In cases where the reservoir is not removable from the patch-like medical device, it is known in the prior art to use a separate transfer syringe to first suck the liquid out of a pre-filled container and then inject the liquid into the reservoir. This approach however has the disadvantage of requiring a separate transfer syringe, which has to be properly sterilized. Furthermore, liquid may get wasted in the transfer if the user, e.g. the patient, is not properly trained or does not have the motoric skills to handle the syringe properly.

### SUMMARY OF THE INVENTION

In a first aspect, it is therefore an object of the present invention to provide a filling aid that enables the reservoir of a patch pump to be filled with a liquid in an easy and quick manner which is hygienically safe.

This first object is achieved by a filling aid assembly according to claim 1. Further embodiments of the invention are laid down in the dependent claims.

The invention provides a filling aid assembly for filling a liquid into a reservoir of a patch pump comprising a piston arranged within the reservoir, the piston being movable to push fluids out of the reservoir by a displacement in a forward direction and to allow fluids to fill the reservoir by a displacement in a backward direction, the filling aid assembly comprising:
a lid part configured to be arranged on top of the patch pump, the lid part having an opening for accessing the reservoir when the lid part is arranged on top of the patch pump to enable fluidic exchange between the reservoir and a pre-filled container containing the liquid;
a dial ring surrounding the lid part and being rotatable with respect to the lid part, and
a transmission gear assembly arranged within the lid part,
wherein the dial ring comprises an internal gearing.

The transmission gear assembly is in engagement with the internal gearing of the dial ring and the transmission gear assembly comprises an output element for connection with a drive mechanism of the patch pump to translate a rotational movement of the dial ring into the displacement of the piston in the forward and/or backward direction within the reservoir.

Especially since the patch pump is intended to be a wearable device, the drive mechanism of the patch pump may be small and delicate. By providing a filling aid that comprises a dial ring and a transmission gear assembly according to the invention, handling becomes easier and the risk of damaging the drive mechanism of the patch pump is minimized, since the drive mechanism of the patch pump does not need to be directly accessed by the user to move the piston.

The filling aid assembly may further comprise a holder configured to receive the pre-filled container containing the liquid, the pre-filled container being in particular a vial or a cartridge, wherein the holder may comprise a hollow needle defining a lumen, the needle having a pointed first end for piercing the pre-filled container, and wherein the holder may have a holder insertion portion being insertable into the opening of the lid part to engage with the patch pump to enable fluidic exchange between the reservoir and the pre-filled container via the lumen.

The holder may comprise a holder base, and an adapter configured to receive a cartridge having a displaceable plug closing the cartridge at a first end. The adapter may comprise a safety cover to prevent the plug from getting ejected when the cartridge is received in the adapter. Such a safety cover may be particularly useful during a first phase of the filling process, where a gas like air may be pushed into the cartridge to create positive pressure to then facilitate the extraction of the liquid in a second phase of the filling process. Without the safety cover, the plug may get ejected from the cartridge due to the positive pressure, potentially causing harm to the user and/or causing spilling of the liquid contained in the cartridge.

Furthermore, the adapter may be removable from the holder base to allow for the holder base to receive a vial having a larger diameter than the cartridge when the adapter is removed. Providing a removable adapter allows for a greater flexibility regarding the choice of pre-filled containers that may be used by a user to fill the reservoir of the patch pump. Preferably, the adapter is reusable.

Preferably, the needle is fixedly attached to the holder base. In such a case, the holder base may be intendent for single use and thus be disposable.

The liquid contained in the pre-filled container may in particular be a solution comprising insulin. In the present disclosure, the terms “vial” and “cartridge” both refer to flasks closed at a first end by pierceable septum. A “vial” is understood to be closed closed-bottomed. A “cartridge” is used in the present context to describe an open-bottomed cylindrical flask with a removable plug closing the second end of the flask. A vial may have a larger diameter than a cartridge.

Prior to filling the liquid into the reservoir, a fluid that may already be contained in the reservoir, in particular a gas, more specifically air or an inert gas like nitrogen or argon, may be pushed out to create a positive pressure in the pre-filled container when the latter is in fluidic connection with the reservoir to facilitate the subsequent extraction of the liquid.

The lid part preferably has a window through which the reservoir is visible when the lid part is arranged on top of the patch pump. The window may be a cut-out in the lid part, which cut-out may have the shape of the reservoir. It is also conceivable for the cut-out to be covered by a see-through window cover.

The lid part may further comprise marks arranged adjacent to the window indicating different filling volumes of the reservoir when the lid part is arranged on top of the patch pump to aid the user.

The lid part, when arranged on top of the patch pump, protects the patch pump during the filling process. By having an opening in the lid part and a holder for the pre-filled container insertable into said opening, the pre-filled container may be safely connected to the patch pump. Ideally, the holder is inserted into the opening in the lid part prior to placing the pre-filled container into the holder to avoid liquid getting spilled once the septum of the pre-filled container has been pierced by the pointed first tip of the needle.

The filling aid assembly may further comprise a base part, wherein the lid part and the base part are connectable to form a cavity inside which the patch pump can be received.

The base part and/or the lid part may comprise alignment features that allow the base part and the lid part to be connected only when the lid part is in a pre-determined orientation with respect to the base part. These alignment features may be box alignment features, tab features and recesses matching the tab features or any other suitable features.

Ideally, the lid part and the base part form a cavity which is sealed when the parts are connected, so as to prevent dirt from entering the cavity and protect the patch pump.

The base part may be provided as a part of a packaging protecting the patch pump prior to use. The base part may comprise means for securing the patch pump on the base part in a pre-determined orientation with respect to the base part. In particular, the base part may comprise a securing arm configured to be engage with a rim of the patch pump to hold the latter in place.

Preferably, the lid part with the dial ring and/or the base part of the filling aid are reusable.

The lid part may comprise a cover and a support part that are made separately and rigidly connected to each other, wherein the transmission gear assembly is arranged between the support part and the cover. In particular, the transmission gear assembly may be at least partially mounted on the support part. In particular, the transmission gear assembly may be held on the support part by a mounting plate that is screwed onto the support part. The cover preferably serves to protect the transmission gear assembly and preferably comprises the opening for accessing the reservoir when the lid part is arranged on top of the patch pump. The cover and the support part may be connected to each other via a set of screws.

The dial ring may be held between the cover and the support part while being rotatable with respect to the cover and the support part. This prevents the dial ring from disengaging from the lid part and in particular prevents the internal gearing of the dial ring from disengaging from the transmission gear assembly.

The dial ring preferably has a peripheral outer surface that is that is configured to be accessible for a hand of a user for manually rotating the dial ring, the peripheral outer surface being preferably textured. In this context, “textured” may refer to any surface modification that facilitates gripping, in particular, the outer surface may be grooved or roughened. Such a textured outer surface provides a good grip when manually operating the dial ring.

The transmission gear assembly may comprise a safety clutch configured to decouple the rotational movement of the dial ring from the displacement of the piston when a torque above a pre-defined torque threshold is applied to the dial ring. As already mentioned, the drive mechanism of the piston in the patch pump may be delicate and may be prone to damage when excessive torque is being applied to it. The safety clutch prevents said excessive torque from damaging the drive mechanism. The user therefore may not have to worry about having to self-regulate the amount of torque applied to the dial ring, which facilitates handling.

The safety clutch may comprise a first gear wheel and a second gear wheel, the first gear wheel and the second gear wheel sharing a common central axis and being arranged on top of each other along said common axis so as to form a frictional connection, wherein the first gear wheel is preferably in engagement with the internal gearing of the dial ring.

Preferably, the first gear wheel and the second gear wheel are configured to rotate in unison due to the frictional connection as long as the torque applied to the dial ring is below or equal to the pre-defined torque threshold, and the first gear wheel is configured to slip with respect to the second gear wheel when the torque applied to the dial ring is above the pre-defined torque threshold.

The safety clutch may comprise a shaft extending along the common central axis, the first gearwheel and the second gearwheel being arranged on the shaft. The shaft may have an end cap at a first end and a threaded portion at a second end, wherein the first gear wheel may have a recess in which the end cap may be received so as to rotate with the first gear wheel. The safety clutch may further comprise at least one nut being screwed onto the threaded portion, thereby pressing the second gear wheel against the first gear wheel to create the frictional connection. In order to ensure a consistent pre-defined torque threshold, the safety clutch may further comprise tensioning means arranged on the shaft between the at least one nut and the second gearwheel, in particular at least one disc spring, preferably combined with a keyed washer.

The output element of the transmission gear assembly may be an output gear mechanism being in engagement with the second gear wheel of the safety clutch, wherein the output gear mechanism is configured to engage with the drive mechanism of the patch pump to enable the displacement of the piston within the reservoir.

The output gear mechanism may comprise an output gear wheel being in engagement with the second gear wheel of the safety clutch, and a sliding sleeve extending perpendicularly from a surface of the output gear wheel. The sliding sleeve may be attached, preferably resiliently attached via a spring, to the output gear wheel. Preferably, the sliding sleeve is configured to receive a pin of the drive mechanism of the patch pump, such that when the pin is received in the sliding sleeve, the pin rotates in unison with the output gear wheel to drive the drive mechanism. Preferably, the pin has a cross-section that is star-like or that has any other shape suitable to handle high torque, in particular any shape that is not circularly symmetric and therefore enables a form fit in circumferential direction, and the sliding sleeve has a receiving portion with a matching cross-section. The spring preferably allows the sliding sleeve to retract when the lid part is placed on top of the patch pump to ensure that the output gear mechanism does not prevent the lid part from being fully assembled onto the base part, even when the cross-section of the pin is initially not aligned with the cross-section of the receiving portion of the sliding sleeve.

In a second aspect, it the present invention provides a medical assembly that comprises:
the filling aid assembly as described above;
a patch pump for delivering a liquid through a patient's skin, the patch pump comprising
a reservoir for containing a liquid;
a piston arranged within the reservoir, the piston being movable to push fluids out of the reservoir by a displacement in a forward direction and to allow fluids to fill the reservoir by a displacement in a backward direction, and
a drive mechanism, in particular piston gear system, for driving the piston.

The transmission gear assembly of the filling aid is in engagement with the drive mechanism to translate a rotational movement of the dial ring into the displacement of the piston within the reservoir in the forward- and/or backward-direction.

The reservoir of the patch pump may comprise an input nozzle, wherein the needle is received inside the input nozzle when the holder insertion portion is inserted in the opening of the lid part, and wherein the insertion portion is preferably configured to connect with the input nozzle via a releasable snap-fit connection. In particular, the holder insertion portion may comprise cantilever arms which engage with a notch on the input nozzle. The input nozzle may be covered by a nozzle septum. The needle may have a pointed second end for piercing through said nozzle septum.

The reservoir may have a toroidal shape, whereby the piston preferably moves within the reservoir along a circle, wherein the circle and the dial ring are concentric when the filling aid assembly and the patch pump are assembled. Such a toroidal shape may enable a compact arrangement of the components in the patch pump, leading to a small overall size, which increases the comfort for a patient wearing the patch pump.

In a preferred embodiment, rotating the dial ring in a first rotational direction, in particular clockwise, causes the piston to move in the forward direction and thereby push fluids, in particular, a gas, more specifically air or an inert gas, out of the reservoir before being filled with the liquid, thereby in particular causing a positive pressure in a pre-filled container containing the liquid that is in fluidic exchange with the reservoir, and wherein rotating the dial ring in a second rotational direction opposite to the first rotational direction, in particular anti-clockwise, causes the piston to move backward and thereby allow the liquid to flow through the lumen of the needle into the reservoir when the pre-filled container is arranged so as to be in fluidic exchange with the reservoir. In particular, the backward movement of the piston may allow the liquid to flow through the lumen of the needle into the reservoir by the action of the positive pressure that has previously been created in the pre-filled container by the forward movement of the piston. In addition or in the alternative, the backward movement of the piston may cause a negative pressure in the reservoir, which sucks the liquid through the lumen of the needle into the reservoir. In order to eliminate gas bubbles in the liquid that has been filled into the reservoir, the dial ring may be slowly rotated back and forth in the second and first rotational directions, respectively, to cause small backward and forward displacements of the piston. To optimally prepare the piston for a subsequent process comprising a delivery of at least a portion of the liquid to a user, said backward and forward displacements of the piston preferably end with a displacement in the forward direction.

Preferably, reservoir is transparent or semi-transparent to allow for a visual detection of gas bubbles in the liquid that has been filled into the reservoir.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a partially exploded perspective view of a medical assembly comprising a filling aid assembly according to an embodiment of the present invention;

FIG. 2 shows a first perspective view of a holder base according to the embodiment of FIG. 1;

FIG. 3 shows a second perspective view of the holder base of FIG. 2;

FIG. 4 shows a front view of the holder base of FIG. 2;

FIG. 5 shows a patch pump;

FIG. 6 shows a sectional view of the medical assembly of FIG. 1 in an assembled state along the sectional plane A-A marked in FIG. 11;

FIG. 7 shows a top view of a lid part with a dial ring according to the embodiment of FIG. 1;

FIG. 8 shows a side view of the lid part with the dial ring of FIG. 7;

FIG. 9 shows a perspective view of the lid part with the dial ring of FIG. 7;

FIG. 10 shows a perspective view of the medical assembly of the according to the embodiment of FIG. 1 in an assembled state without the holder;

FIG. 11 shows a top view of the embodiment of FIG. 10;

FIG. 12 shows an exploded perspective view of the lid part, the dial ring and the transmission gear assembly of the embodiment of FIG. 1;

FIG. 13 shows a top view of the patch pump according to the embodiment of FIG. 5;

FIG. 14 shows a perspective view of the patch pump according to the embodiment of FIG. 5 and an exploded perspective view of an output gear mechanism;

FIG. 15 shows a cross-sectional view of a safety clutch according to the embodiment of FIG. 1;

FIG. 16 shows an exploded perspective view of the safety clutch of FIG. 15;

FIG. 17 shows a cross-sectional view of an output gear mechanism according to the embodiment of FIG. 1;

FIG. 18 shows an exploded perspective view of the output gear mechanism of FIG. 17;

FIG. 19 shows a sectional view of the dial ring, the safety clutch and the output gear mechanism according to the embodiment of FIG. 1;

FIG. 20 shows an exploded sectional view of the dial ring, the safety clutch and the base part according to the embodiment of FIG. 1, and

FIG. 21 shows a sectional view of the embodiment of FIG. 6 along the sectional plane B-B marked in FIG. 11.

### DESCRIPTION OF PREFERRED EMBODIMENTS

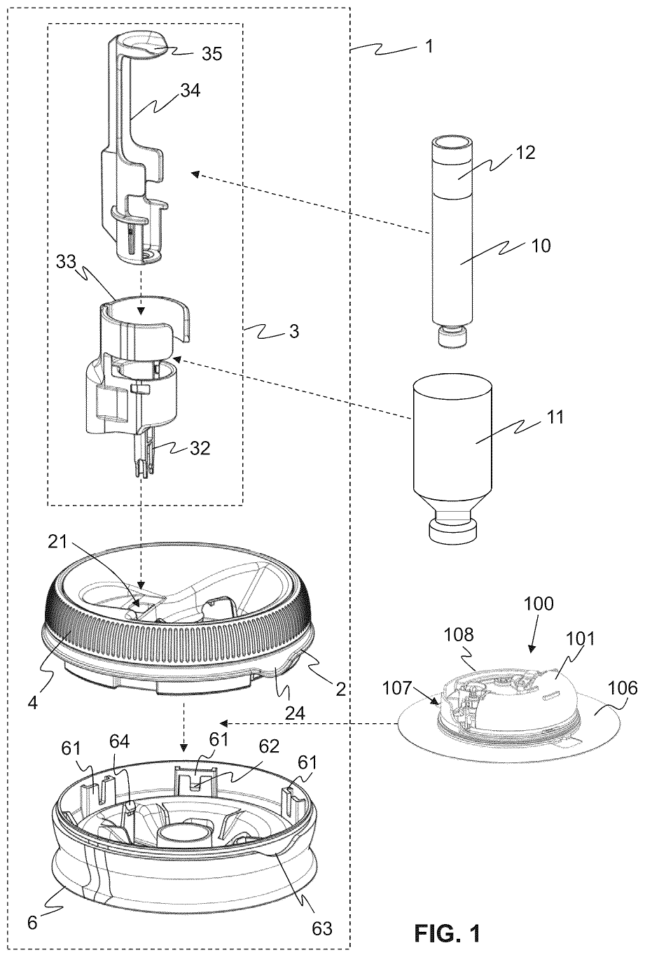

FIG. 1 illustrates a medical assembly according to an embodiment of the present invention. The medical assembly comprises a filling aid assembly 1 and a patch pump 100.

The filling aid assembly 1 comprises a lid part 2 and a base part 6, which are connectable to form a cavity inside which a patch pump 100 having a reservoir 101 to be filled can be received as indicated by the dashed arrow in FIG. 1. The filling aid assembly 1 further comprises a dial ring 4 surrounding the lid part 2, the dial ring 4 being rotatable with respect to the lid part 2. The lid part 2 has an opening 21 for accessing the reservoir 101 of the patch pump 100 when the lid part 2 is arranged on top of the patch pump 100. In order to be able to fill the reservoir 101 through said opening 21, the opening 21 needs to be properly aligned with respect to the reservoir 101. When assembling the present embodiment of the medical assembly, the patch pump 100 is first placed onto the base part 6 in correct orientation. For this purpose, the base part 6 may comprise a securing arm 64 and the patch pump 100 may comprise a plaster 106 attached to a bottom surface of the patch pump 100, wherein the plaster 106 has an alignment hole 107 that is to be aligned such that the securing arm 64 can reach through the alignment hole 107 of the plaster and engage with a rim 108 of the patch pump 100 to hold the patch pump 100 in place on the base part 6.

In order to then facilitate alignment of the lid part 2 with respect to the base part 6 and thus also to the patch pump 100 with the reservoir 101, the base part 6 may comprise box alignment features 61 distributed along an inner rim of the base part 6. Preferably, at least one the box alignment features 61 comprises a snap arm 62 to make a “click” on engagement with the lid part 2 to inform the user that the lid part 2 and the base 6 have been properly connected. Furthermore, the lid part 2 may comprise a tab feature 24 that engages with a corresponding cut-out 63 of an outer rim of the base part 6.

The filling aid assembly 1 further comprises a holder 3 for receiving a pre-filled container that contains the liquid to be filled into the patch pump 100. The pre-filled container is closed at a first end by a pierceable septum. The holder 3 shown here comprises a holder base 33 and an adapter 34. The holder base 33 is configured to directly receive vials 11 with a certain standardized vial diameter, while the adapter 34 may be inserted into the holder base 33 to receive pre-filled containers, in particular cartridges 10, having a smaller diameter than said standardized vial diameter. The adapter 34 shown in FIG. 1 is particularly suitable for receiving cartridges that are closed at their first end by a pierceable septum and at their second end by a plug 12, as it comprises a safety cover 35 which covers the second end of the cartridge 10 when the cartridge is in the adapter 34, thereby preventing the plug from getting ejected from the cartridge 10 when a gas like air is being pumped into the cartridge prior to extracting the liquid contained in the cartridge 10. In particular, the adapter 34 may have a receiving portion configured to receive and hold the cartridge 10, and an arm portion extending from said receiving portion along the cartridge 10 when the latter is placed in the adapter 34. The safety cover 35 may protrude from the arm portion at an angle so as to extends over the second end of the cartridge 10 parallel to said second end when the cartridge 10 is in the adapter 34, such that the second end is covered. The adapter 34 may be configured to surround the cartridge 10 only partially in a circumferential direction, i.e. the adapter 34 may be open to one side in order for the cartridge to be insertable into the adapter from said one side, i.e. in a direction parallel to the safety cover, as shown in FIG. 1.

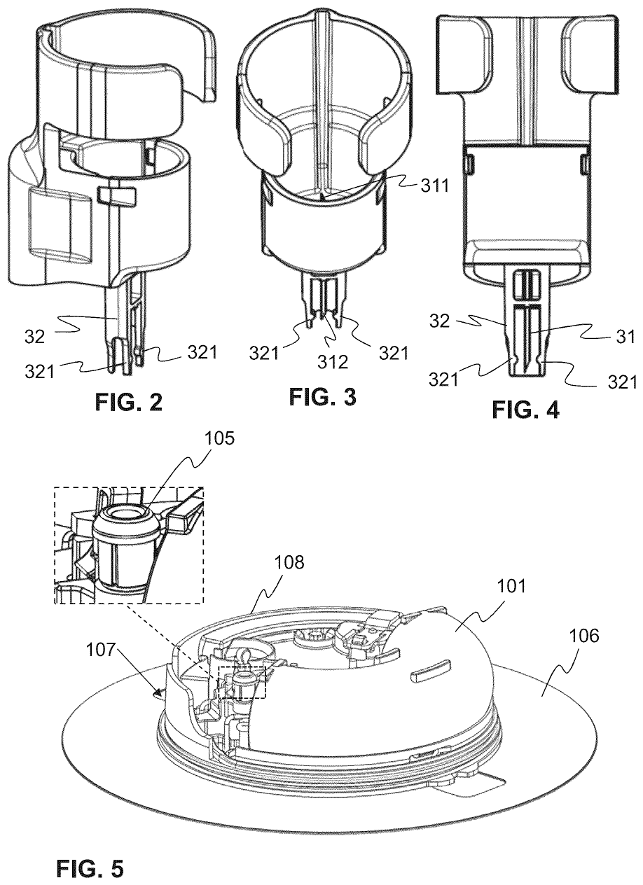

The holder 3, in particular the holder base 33, has an insertion portion 32 that is insertable into the opening 21 of the lid part 2. An enlarged view of the holder base 33 is shown in FIGS. 2-4, wherein FIGS. 2 and 3 are perspective views and FIG. 4 is a front view of the holder base 33. The holder base 33 comprises a hollow needle 31 defining a lumen. The needle 31 has a pointed first end 311 arranged to pierce the septum of the pre-filled container and a pointed second end 312 arranged in the insertion portion 32. FIG. 5 shows a perspective view of the patch pump 100. The reservoir 101 comprises an input nozzle 105, an enlarged view of which can be seen in the inset of FIG. 5. The input nozzle 105 has an essentially cylindrical shape extends vertically upwards in a direction that is perpendicular to the displacement direction of the piston 102 in the reservoir 101, and is arranged at an end of the reservoir 101 and fluidically connected to the latter. The insertion portion 32 has cantilever arms 321 and the input nozzle 105 has matching indents, which enables the insertion portion 32 to be connected with the input nozzle 105 in a releasable snap-fit manner when the holder insertion portion 32 is inserted in the opening 21 of the lid part 2. The input nozzle 105 may be covered by a septum, in which case the pointed second end 312 of the needle pierces through said septum when the insertion portion 32 is engaged with the input nozzle 105. The pointed second end 312 of the needle 31 preferably does not protrude beyond the cantilever arms 21 to prevent damage of the needle 31 as well as injuries of the user.

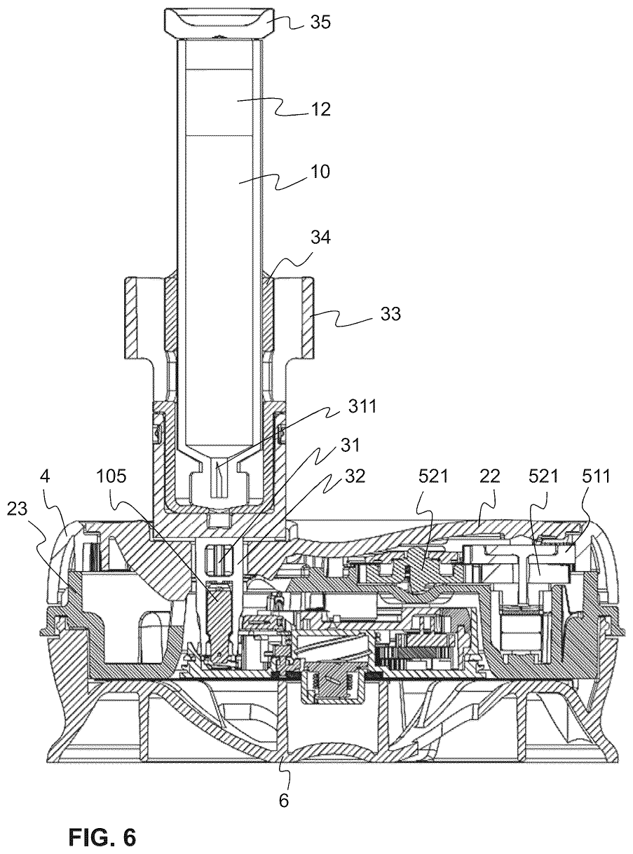

FIG. 6 shows a sectional view along the sectional plane A′-A′ (shown in FIG. 11) of the medical assembly of FIG. 1 with all parts being assembled. The pointed first end 311 of the needle 31 reaches into the cartridge 10 that is arranged in the adapter 34 of the holder 3, while the pointed second end 312 of the needle reaches into the input nozzle 105 of the reservoir 101 to enable fluidic exchange between the cartridge 10 and the reservoir 101.

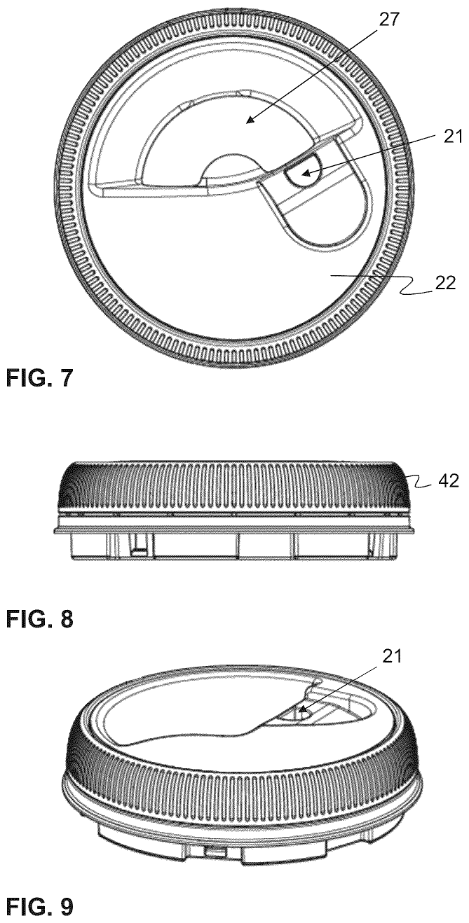

FIGS. 7-9 show the lid part 2 together with the dial ring 4 surrounding the lid part 2. FIG. 7 shows a top view, where the opening 21 is clearly visible, while FIG. 8 shows a side view and FIG. 9 shows a perspective view. The lid part 2 further has a window 27 through which the reservoir is ideally visible when the lid part 2 is arranged on top of the patch pump. The window 27 may be a cut-out in the lid part 2, which cut-out has the shape of the reservoir 101, as shown in FIG. 7. It is also conceivable for the cut-out to be covered by a see-through window cover (not shown here).

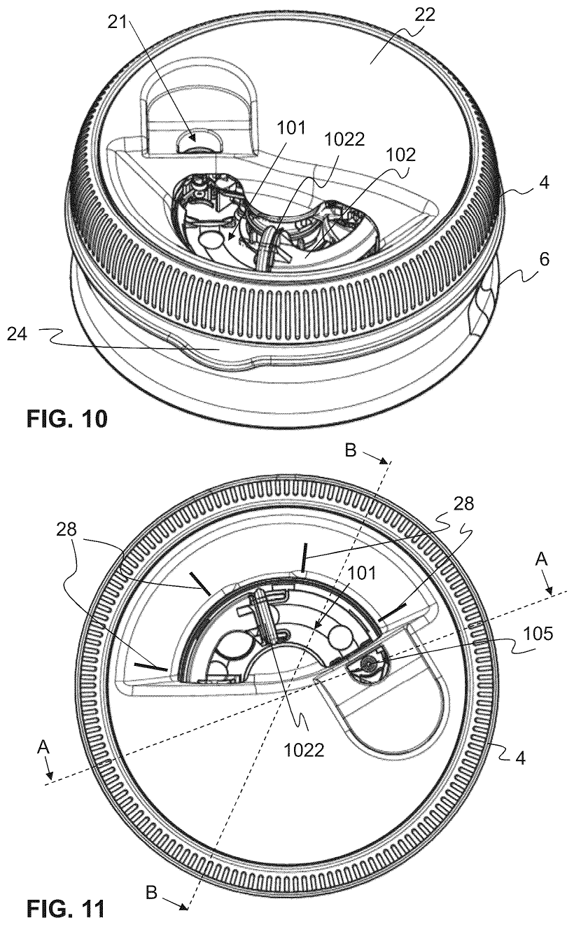

The medical assembly of FIG. 1 without the holder 3 is shown in a perspective view in FIG. 10 and in a top view in FIG. 11. As can be seen in FIG. 10 and FIG. 11, the patch pump comprises a piston 102 arranged within the reservoir 101. The piston 102 is movable to push fluids out of the reservoir 101 through via input nozzle 105 by a displacement in a forward direction and to allow fluids to fill the reservoir 101 by a displacement in a backward direction. By rotating the dial ring 4, the piston 102 in the reservoir 101 may be moved backward and forward. Preferably, the piston 102 comprises a piston head 1022 having a cross-section matching the cross-section of the reservoir 101 and which comprise a seal element surrounding the piston head 1022, the seal element gliding along an inner wall of the reservoir 101 when the piston head 1022 is moved backward and forward, thereby sealing the reservoir 101 at one end.

In the preferred embodiment shown here, the reservoir has a toroidal shape, the piston 102 therefore moving within the reservoir 101 along a circle, wherein the circle and the dial ring 4 are concentric.

Preferably, rotating the dial ring 4 in a clockwise direction causes the piston 102 to move in the forward direction and thereby push gas, in particular, air of the reservoir 101 through the lumen of the needle 31 into the pre-filled container 10,11 when the latter is installed in the holder 3 to create positive pressure in the pre-filled container. Rotating the dial ring 4 in an anti-clockwise direction then causes the piston 102 to move backward and thereby create negative pressure so as to allow the liquid to flow out of the pre-filled container 10,11 through the lumen of the needle 31 into the reservoir 101. Preferably, the reservoir 101 is transparent or semi-transparent as shown in FIG. 10 and FIG. 11, so that the piston 102 and the filling state may be observed by the user through the window 27 in the cover 22 of the lid part 2. In case the user observers gas bubbles in liquid that has been filled into the reservoir 101, the user may slowly rotate the dial ring back and forth to cause small backward and forward displacements of the piston in order to remove the bubbles. Said reciprocating displacements of the piston preferably end with a displacement in the forward direction. Ending with a displacement in the forward direction causes the seal element surrounding the piston head 1022, which may be made of a flexible material, to be oriented in a defined starting position, which enables a more reliable and precise delivery of a first shot of the liquid to be administered to a user than if the seal element is left in an undefined distorted state after a displacement in the backward direction.

In some embodiments, marks 28 may be arranged on the lid part 2 adjacent to the window 27, in particular on the cover 22 of the lid part 2 as shown in FIG. 11, indicating different filling volumes to a user. Alternatively, the marks 28 may also be arranged on the reservoir 101.

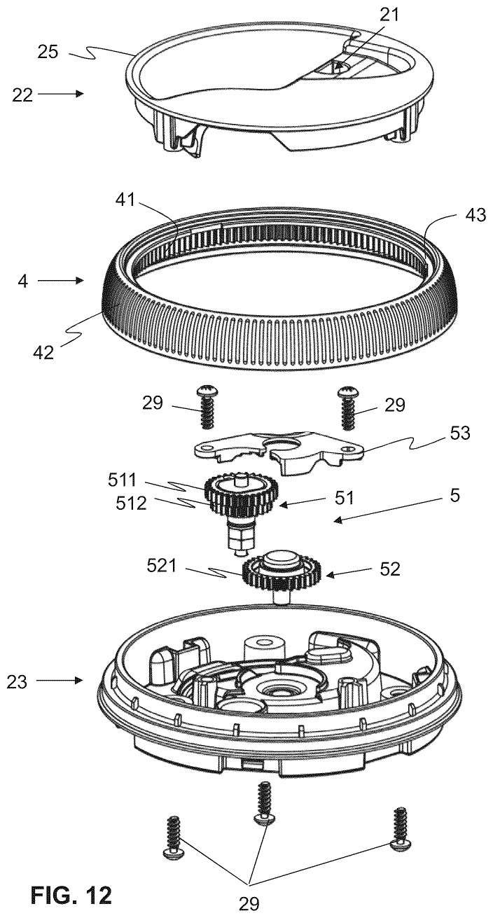

FIG. 12 shows an exploded view of the lid part 2 together with the dial ring 4 and a transmission gear assembly 5. The lid part 2 comprises a cover 22 and a support part 23 that are connected to each other in an assembled state, in particular via screws 29. The cover 22 comprises the opening 21 into which the holder insertion portion 32 can be inserted. The dial ring 4 is held between the cover 22 and the support part 23 and is rotatable with respect to the cover 22 and the support part 23. In particular, the cover 22 has a cover rim 25 which rests on an inner rim 43 of the dial ring 4 when the lid part is assembled, the inner rim 43 protruding radially inwards.

In this preferred embodiment, the dial ring has a peripheral outer surface 42 that is textured, in particular exhibiting a plurality of grooves arranged next to each other around the entire peripheral outer surface 42, to facilitate grasping of the dial ring 4 in order to rotate it with one hand while preferably holding onto the base part 6 with the other hand.

The dial ring 4 comprises an internal gearing 41 facing radially inwards, which is in engagement with the transmission gear assembly 5. The transmission gear assembly 5 is arranged between the support part 23 and the cover 22. As shown in FIG. 12, the transmission gear assembly may be mounted on the support part 23 and held in place by a mounting plate 53 that is screwed onto the support part 23 using screws 29.

The transmission gear assembly 5 comprises an output element for connection with a drive mechanism of the patch pump to translate a rotational movement of the dial ring 4 into the displacement of the piston 102 in the forward and/or backward direction within the reservoir 101.

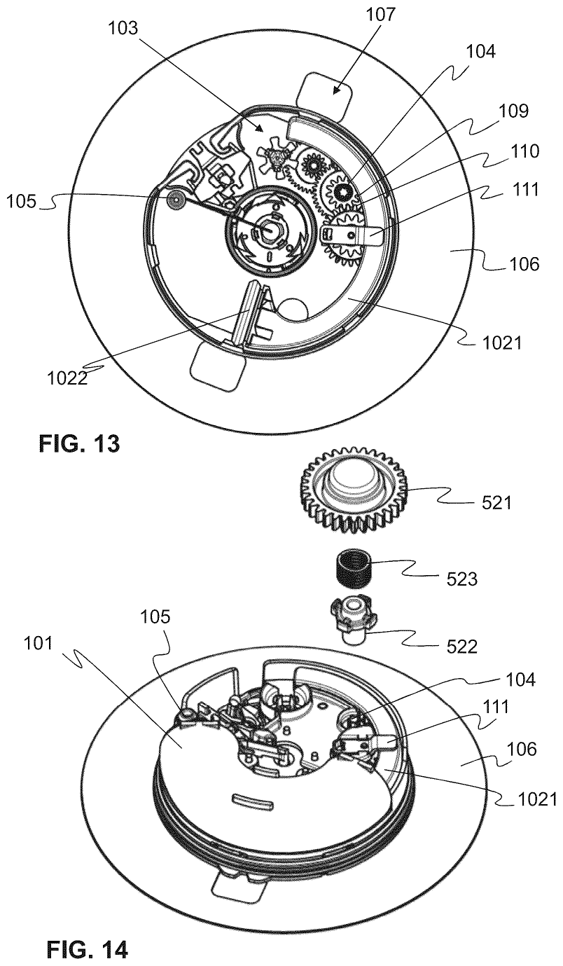

In the embodiment shown here, said output element is an output gear mechanism 52 which is configured to engage with a piston gear system 103 of the patch pump 100 to enable the displacement of the piston 102 within the reservoir 101. The piston gear system 103 of the patch pump 100 is visible in a top view in FIG. 13.

In FIG. 14, the patch pump 100 is shown in a perspective view together with the output gear mechanism 52. In addition to the output gear wheel 521, the output gear mechanism 52 further comprises a sliding sleeve 522 extending perpendicularly from a surface of the output gear wheel 521, the sliding sleeve 522 being resiliently attached to the output gear wheel via a spring 523 and configured to slide along the axis of rotation of the output gear wheel 521. The piston gear system 103 of the patch pump 100 comprises a pin 104 and the sliding sleeve 522 is configured to receive the pin 104, in particular establishing a form fit with respect to the circumferential direction, such that the pin 104 rotates in unison with the output gear wheel 521 to drive the piston gear system 103. The pin 104 in the embodiment shown here has a cross-section that is star-like and the sliding sleeve 522 has a receiving portion with a matching cross-section. The spring 523 allows the sliding sleeve 522 to retract when the lid part 2 is placed on top of the patch pump 100 to ensure that the output gear mechanism 52 will not prevent the lid part 2 from being fully assembled onto the base part 6, even when the cross-section of the pin 104 is initially not aligned with the cross-section of the receiving portion of the sliding sleeve 522. In such a case, once the lid part 2 and the base part 6 are assembled, the receiving portion of the sliding sleeve 522 might first be slightly rotated by rotating the dial ring 4 until the cross-sections are aligned and the sliding sleeve 522 slides onto the pin 104.

As shown in FIG. 13, the pin 104 may be attached to a first piston gear wheel 109, which in turn may transmit its rotation to at least a second piston gear wheel 110, the second piston gear wheel 110 being in engagement with an internal gearing (not visible here) of an arm 1021 of the piston 102 to translate the rotation of the second piston gear wheel 110 into the circular displacement of the piston 102. The arm 1021 of the piston 102 extends beyond the reservoir 101 along the rim 108 of the patch pump 100, following the curvature of said rim 108. A clip element 111 may be arranged on the arm 1021 of the piston 102 in order to retain the arm 1021.

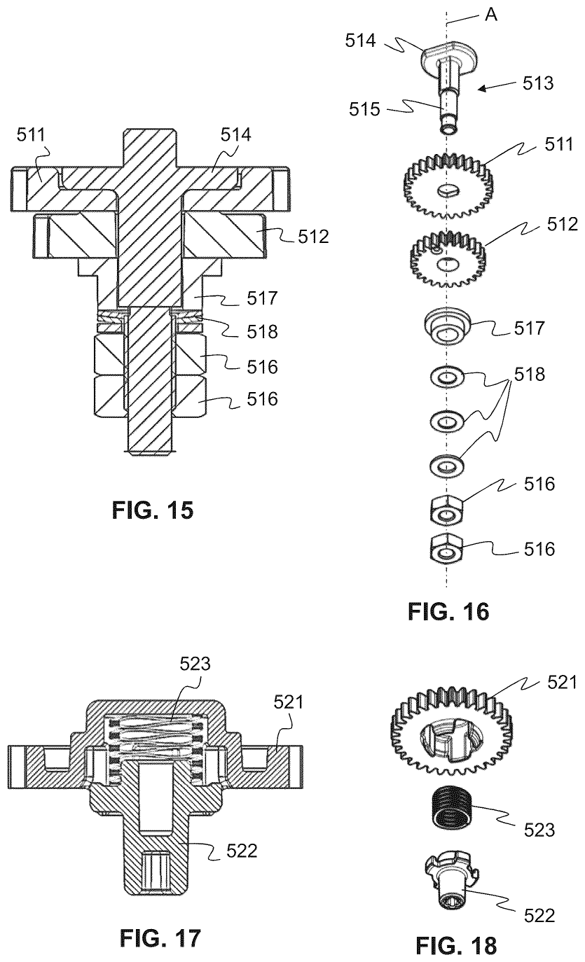

As shown in FIG. 12, as well as in greater detail in FIG. 15 and FIG. 16, the transmission gear assembly 5 comprises a safety clutch 51 that is configured to decouple the rotational movement of the dial ring 4 from the displacement of the piston 102 when a torque above a pre-defined torque threshold is applied to the dial ring 4.

FIG. 15 shows a sectional view of the safety clutch 51, while FIG. 16 shows said safety clutch 51 in an exploded view. The safety clutch 51 comprises a first gearwheel 511 and a second gear wheel 512, the first gear wheel and the second gear wheel 512 sharing a common central axis A and being arranged on top of each other along said common axis A. In the present embodiment, the first gear wheel 511 has a larger diameter than the second gear wheel 512 and is in direct engagement with the internal gearing 41 of the dial ring 4, while the second gear wheel 512 is in engagement with the output gear wheel 521 of the output mechanism 52.

A sectional view of the output mechanism 52 is shown in FIG. 17, while FIG. 18 shows an exploded view of the output mechanism 52 with the output gear wheel 521, the sliding sleeve 522 and the spring 523.

The safety clutch 51 comprises a shaft 513 extending along the common central axis, the shaft 513 having an end cap 514 at a first end and a threaded portion 515 at a second end. Towards the first end (i.e. where there is no thread), the shaft has a D-shaped cross-section. The first gear wheel 511 and the second gear wheel 512 are arranged on the shaft 513. The first gear wheel 511 has a recess in which the end cap is received. Said end cap may have at least one straight edge or another key feature a matching a corresponding shape of the recess such that the end cap is forced to rotate with the first gear wheel 511 when received in the recess. The first gear wheel 511 furthermore has a D-shaped center hole form-fitted to the cross-section of the shaft 513 such that the first gear wheel 511 rotates in unison with the shaft 513. The second gear wheel 512 however has a center hole in which the shaft may freely rotate, hence, a rotation of the shaft does not automatically entrain a rotation of the second gearwheel 512. The safety clutch 51 comprises at least one nut 516, in the embodiment shown in FIG. 13 two nuts 516, that are screwed onto the threaded portion of the shaft 515 to press the second gear wheel 512 against the first gear wheel in order to create a frictional connection between the first gear wheel 511 and the second gear wheel 512. To improve tensioning, the safety clutch 51 may comprise a keyed washer 517 with a D-shaped hole, as well as disc springs 518 arranged on the shaft 513 between the second gear wheel 512 and the nuts 516. As a result of said frictional connection, the second gear wheel 512 rotates in unison with the first gear wheel 511 as long as the torque applied to the dial ring 4 is below or equal to the pre-defined torque threshold. The pre-defined toque threshold depends on the force with which the first gear wheel 511 and the second gear wheel are pressed against each other, i.e. in this case by how tight the nuts 516 are screwed. When the torque applied to the dial ring 4 and transmitted to the first gear wheel 511 is above the pre-defined torque threshold, the first gear wheel 511 slips with respect to the second gear wheel 512, the rotation is no longer transmitted to the second gear wheel 512. Since the second gear wheel 512 is in engagement with the output gear wheel 521, the rotational movement of the dial ring will thus also no longer be translated into a displacement of the piston 102 of the patch pump 100. The safety clutch 51 thus prevents too much torque from being applied to the piston gear system 103, protecting the latter from potential damage.

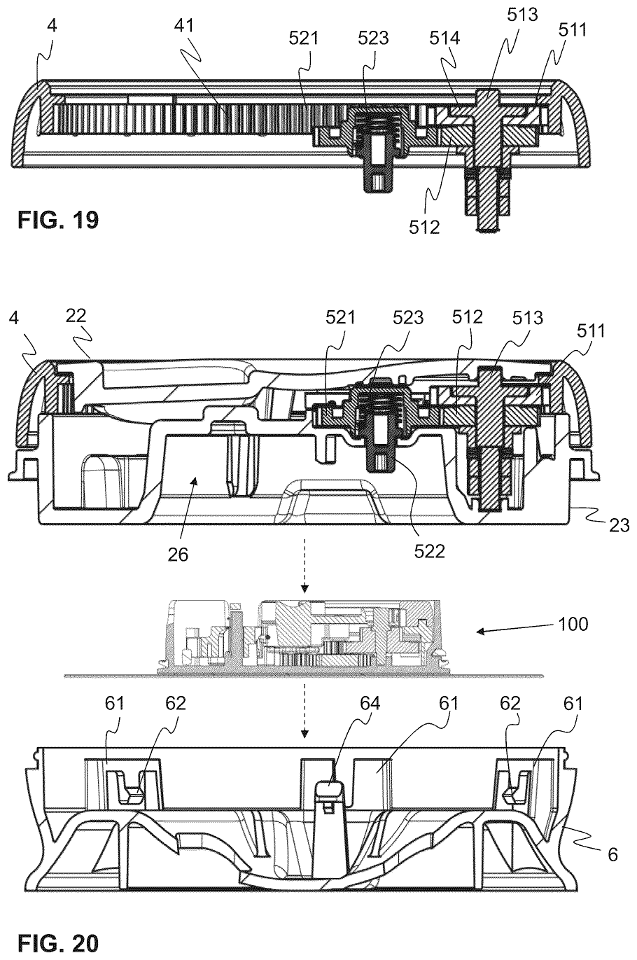

FIG. 19 shows a sectional view of the dial ring 4, the safety clutch 51 and the output gear mechanism 52, where the engagement of the first gear when 511 with the internal gearing 41 of the dial ring 4 is visible, as well as the engagement of the second gear wheel 512 with the output gear wheel 521.

FIG. 20 shows the lid part 2, the dial ring 4, the patch pump 100 and the base part 6 in a sectional view. The dashed arrows indicate how the parts are being assembled. As can be seen in this sectional view, the transmission gear assembly 5 is essentially arranged on the support part 23 of the lid part 2, with the sliding sleeve 522 of the output gear mechanism 52 protruding into a recess 26 defined by the support part 23, which recess 26, together with the base part 6, forms the cavity in which the patch pump 100 is received when the lid part 2 and the base part 6 are connected. Furthermore, the snap arms 62 and the securing arm 64 of the base part 6 already described above are visible in this sectional view.

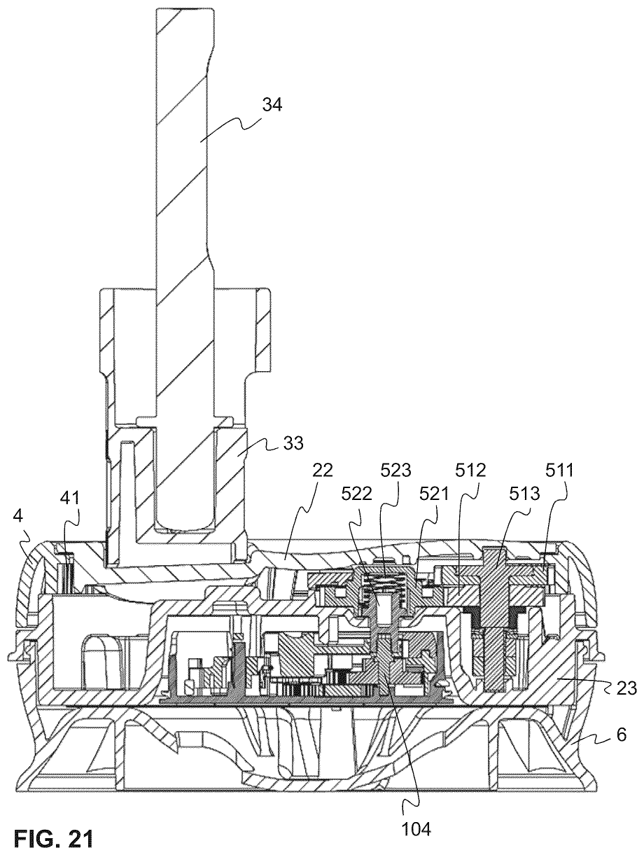

FIG. 21 shows a sectional view of the medical assembly of FIG. 6, however at a different angle, i.e. along the sectional plane B-B marked in FIG. 11, to show the transmission mechanism 5 being in engagement with the piston gear system 103 of the patch pump 100. In particular, FIG. 21 shows the pin 104 of the piston gear system 103 being received in the sliding sleeve 522 of the output gear mechanism 52, hence depicting how rotating the dial ring 4 causes a rotation of the first gear wheel 511 of the clutch 51, which, provided that the torque is above the pre-defined threshold as described above, entrains a rotation of the second gear wheel 512 due to the frictional connection, the rotation of the second gear wheel 512 in turn causing a rotation of the output gearwheel 521 and therefore of the sliding sleeve 522, which then drives the displacement of the piston by rotating the pin 104.

LIST OF REFERENCE SIGNS

| | | | |
|---|---|---|---|
| 1 | filling aid assembly | 516 | nut |
| 2 | lid part | 517 | keyed washer |
| 21 | opening | 518 | disc spring |
| 22 | cover | 52 | output gear mechanism |
| 23 | support part | 521 | output gear wheel |
| 24 | tab feature | 522 | sliding sleeve |
| 25 | cover rim | 523 | spring |
| 26 | recess | 53 | mounting plate |
| 27 | window | 6 | base part |
| 28 | marks | 61 | box alignment features |
| 29 | screws | 62 | snap arm |
| 3 | holder | 63 | cut-out |
| 31 | hollow needle | 64 | securing arm |
| 311 | pointed first end | 10 | cartridge |
| 312 | pointed second end | 11 | vial |
| 32 | holder insertion portion | 12 | plug |
| 321 | cantilever arms | 100 | patch pump |
| 33 | holder base | 101 | reservoir |
| 34 | adapter | 102 | piston |
| 35 | safety cover | 1021 | piston arm |
| 4 | dial ring | 1022 | piston head |
| 41 | internal gearing | 103 | piston gear system |
| 42 | peripheral outer surface | 104 | pin |
| 43 | inner rim | 105 | input nozzle |
| 5 | transmission gear assembly | 106 | plaster |
| 51 | safety clutch | 107 | alignment hole |
| 511 | first gear wheel | 108 | rim |
| 512 | second gear wheel | 109 | first piston gear wheel |
| 513 | shaft | 110 | second piston gear wheel |
| 514 | end cap | 111 | clip element |
| 515 | threaded portion | | |

1. A filling aid assembly for filling a liquid into a reservoir of a patch pump comprising a piston arranged within the reservoir, the piston being movable to push fluids out of the reservoir by a displacement in a forward direction and to allow fluids to fill the reservoir by a displacement in a backward direction,
the filling aid assembly comprising:
a lid part configured to be arranged on top of the patch pump, the lid part having an opening for accessing the reservoir when the lid part is arranged on top of the patch pump to enable fluidic exchange between the reservoir and a pre-filled container containing the liquid;
a dial ring surrounding the lid part and being rotatable with respect to the lid part; and
a transmission gear assembly arranged within the lid part,
wherein the dial ring comprises an internal gearing,
wherein the transmission gear assembly is in engagement with the internal gearing of the dial ring, and
wherein the transmission gear assembly comprises an output element for connection with a drive mechanism of the patch pump to translate a rotational movement of the dial ring into the displacement of the piston in the forward and/or backward direction within the reservoir.

2. The filling aid assembly of claim 1, further comprising a holder configured to receive the pre-filled container containing the liquid, the holder comprising a hollow needle defining a lumen, the needle having a pointed first end for piercing the pre-filled container,
wherein the holder has a holder insertion portion being insertable into the opening of the lid part to engage with the patch pump to enable fluidic exchange between the reservoir and the pre-filled container via the lumen.

3. The filling aid assembly of claim 2, wherein the holder comprises
a holder base, and
an adapter configured to receive a cartridge having a displaceable plug closing the cartridge at a first end,
wherein the adapter comprises a safety cover configured to cover the first end of the cartridge to prevent the plug from getting ejected when the cartridge is received in the adapter, and
wherein the adapter is removable from the holder base to allow for the holder base to receive a vial having a larger diameter than the cartridge when the adapter is removed.

4. The filling aid assembly of claim 1, further comprising a base part, wherein the lid part and the base part are connectable to form a cavity configured for receiving the patch pump.

5. The filling aid assembly of claim 1, wherein the lid part comprises a cover and a support part that are made separately and rigidly connected to each other,
wherein the transmission gear assembly is arranged between the support part and the cover.

6. The filling aid assembly of claim 5, wherein the dial ring is held between the cover and the support part while being rotatable with respect to the cover and the support part.

7. The filling aid assembly of claim 1, wherein the transmission gear assembly comprises a safety clutch configured to decouple the rotational movement of the dial ring from the displacement of the piston when a torque above a pre-defined torque threshold is applied to the dial ring.

8. The filling aid assembly of claim 7,
wherein the safety clutch comprises a first gear wheel and a second gear wheel, the first gear wheel and the second gear wheel sharing a common central axis and being arranged on top of each other along said common axis so as to form a frictional connection,
wherein the first gear wheel is in engagement with the internal gearing of the dial ring,
wherein the first gear wheel and the second gear wheel are configured to rotate in unison due to the frictional connection as long as the torque applied to the dial ring is below or equal to the pre-defined torque threshold, and
wherein the first gear wheel is configured to slip with respect to the second gear wheel when the torque applied to the dial ring is above the pre-defined torque threshold.

9. The filling aid assembly of claim 8, wherein the safety clutch comprises a shaft extending along the common central axis, the first gear wheel and the second gear wheel being arranged on the shaft, the shaft having an end cap at a first end and a threaded portion at a second end,
wherein the first gear wheel has a recess in which the end cap is received so as to rotate with the first gear wheel, and
wherein the safety clutch further comprises at least one nut being screwed onto the threaded portion, thereby pressing the second gear wheel against the first gear wheel to create the frictional connection.

10. The filling aid assembly of claim 8, wherein the output element of the transmission gear assembly is an output gear mechanism being in engagement with the second gear wheel of the safety clutch, wherein the output gear mechanism is configured to engage with the drive mechanism of the patch pump to enable the displacement of the piston within the reservoir.

11. The filling aid assembly of claim 10, wherein the output gear mechanism comprises
an output gear wheel being in engagement with the second gear wheel of the safety clutch, and
a sliding sleeve extending perpendicularly from a surface of the output gear wheel and being attached to the output gear wheel,
wherein the sliding sleeve is configured to receive a pin of drive mechanism of the patch pump, such that when the pin is received in the sliding sleeve, the pin rotates in unison with the output gear wheel to drive the drive mechanism of the patch pump.

12. A medical assembly comprising:
the filling aid assembly of claim 1; and
a patch pump for delivering a liquid through a patient's skin, the patch pump comprising:
a reservoir for containing a liquid;
a piston arranged within the reservoir, the piston being movable to push fluids out of the reservoir by a displacement in a forward direction and to allow fluids to fill the reservoir by a displacement in a backward direction;
a drive mechanism for driving the piston;
wherein the transmission gear assembly of the filling aid is in engagement with the drive mechanism of the patch pump to translate a rotational movement of the dial ring into the displacement of the piston within the reservoir in the forward and/or backward direction.

13. The medical assembly of claim 12, wherein the reservoir of the patch pump comprises an input nozzle,
wherein the needle is received inside the input nozzle when the holder insertion portion is inserted in the opening of the lid part.

14. The medical assembly of claim 12, wherein the reservoir has a toroidal shape, whereby the piston moves within the reservoir along a circle, wherein the circle and the dial ring are concentric when the filling aid assembly and the patch pump are assembled.

15. The medical assembly of claim 12, wherein rotating the dial ring in a first rotational direction causes the piston to move in the forward direction and thereby push a gas out of the reservoir before being filled with the liquid and
wherein rotating the dial ring in a second rotational direction opposite to the first rotational direction causes the piston to move backward and thereby allow the liquid to flow through the lumen of the needle into the reservoir when the pre-filled container is arranged so as to be in fluidic exchange with the reservoir.

16. The medical assembly of claim 12, wherein the reservoir is transparent or semi-transparent to allow for a visual detection of gas bubbles in the liquid that has been filled into the reservoir.

17. The filling aid assembly of claim 1, wherein the pre-filled container is a vial or a cartridge.

18. The filling aid assembly of claim 5, wherein the dial ring has a peripheral outer surface that is configured to be accessible for a hand of a user for manually rotating the dial ring.

19. The filling aid assembly of claim 18, wherein the peripheral outer surface is textured.

20. The filling aid assembly of claim 11, wherein the sliding sleeve is resiliently attached to the output gear wheel via a spring.

21. The medical assembly of claim 12, wherein the drive mechanism is a piston gear system.

22. The medical assembly of claim 13, wherein the insertion portion is configured to connect with the input nozzle via a releasable snap-fit connection.

23. The medical assembly of claim 15, wherein the first rotational direction is clockwise and wherein the second rotational direction is anti-clockwise.
